(19) United States
(12) Patent Application Publication
(10) Pub. No.: US 2020/0345287 A1
(43) Pub. Date: Nov. 5, 2020

(54) MENTAL FITNESS ASSESSMENT SYSTEMS AND METHODS USING PHYSIOLOGIC SIGNAL INPUTS

(71) Applicant: WITFIT, Ltd., Oxford (GB)

(72) Inventors: Michael P. H. Lau, Edmonds, WA (US); Gráinne Máire McLoughlin, London (GB)

(21) Appl. No.: 16/861,558

(22) Filed: Apr. 29, 2020

Related U.S. Application Data
(60) Provisional application No. 62/841,056, filed on Apr. 30, 2019.

(51) Int. Cl. A61B 5/16; A61B 5/0478; A61B 5/048; A61B 5/00; A61B 5/0482; A61B 5/04

(52) U.S. Cl. CPC A61B 5/165; A61B 5/0478; A61B 5/048; A61B 5/04012; A61B 5/7267; A61B 5/0482; A61B 5/162

(57) ABSTRACT

A method and system for assessing a mental fitness state of a person using objective physiologic signals from the person includes arranging one or more sensors in proximity to the person's head to measure at least one electroencephalogram (EEG) signal of the person, and analyzing the at least one EEG signal by computing a power in the EEG signal in an EEG signal band; determining a characteristic of the computed power that, for the EEG signal band, is indicative of an emotional or cognitive state of the person; and assessing a mental fitness state of the person based on the determined characteristic. An assessment output is generated which reports the assessed mental fitness state of the person. In at least one implementation, the EEG signal contains oscillating theta waves, and a variance in timing of maximum power occurring in the theta waves around a determined average latency is calculated.

### BACKGROUND

### Technical Field

The present disclosure pertains to systems, methods, and applications for assessing a person's mental fitness, and in particular using physiologic signal inputs, such as electroencephalogram (EEG) signals, in such assessments.

### Description of the Related Art

Mental fitness is a broad term that includes the ability to adapt thinking to accommodate changing internal states and external circumstances, to voluntarily guide behavior in a goal-directed fashion, and ignore internal and external distractions. Essentially, it is a person's ability to be “on task.” A key element of mental fitness is known in the cognitive literature as adaptive, or cognitive, control. This cognitive ability is critical to making good decisions and optimally responding to changing or challenging environments.

Decision-making is a highly developed mental ability in humans. While humans make many decisions throughout life, some that are important or complicated, while others that are trivial or easy, the process of decision-making is rather complex and involves multiple cognitive factors, including attention and memory, and is certainly influenced by emotion. Most of us have at one time or another experienced the phenomenon of being “too angry to speak” or “mumbling nonsense while too excited in trying to impress.” These are examples of instances where possibly cognitive control had been suboptimal and therefore emotion surpassed the necessary cognitive processes for communication. The impact of poor cognitive control over emotional processes has been well-documented in the financial and business worlds. It is well known that emotions affect humans while making economic decisions, often against sound financial principles and reasons, so that emotion outpaces logic and poor decisions may result. Behavioral economics and behavioral finance are disciplines studying the effects of emotion and behavior on economic activities such as investment, consumer preference, and marketing. A number of cognitive processes are impacted by poor self-control, including attention. Lack of attention potentially means missed information, which in turn also affects decision-making.

A person's mental fitness certainly affects their decisions over important issues in their life: marriage and relationships, job and school, or where to settle, for example. It would be ideal if humans could make rational decisions without being overly influenced by, or worse overcome with, emotions or a deficit of attention—which broadly could be classified as being mentally unfit in that moment. It would be of great value if one could recognize the contribution of mental fitness in the decision-making process, thereby enabling one to re-evaluate the validity of the decision. It would be ideal if one can make decisions in an optimal mental fitness state.

Therefore, it would be an innovation to assess mental fitness neurophysiologically and use such assessment to quantitatively determine the person's mental state and thereby predict its influence on the person's decision-making and other cognitive life tasks in the moment. Mental fitness as related to decision-making may become a more critical issue as a person is aging or at the early stage of dementia. Indeed, a recent paper showed that brain waves, or spectral assessment of electro-magnetic brain activity, shows predictive power for early diagnosis of dementia (Priyanka et al., 2018). In such a situation, the person might be able to compensate enough to appear to be functional, but decision-making, especially the intricate decision-making that can be detected more precisely using EEG measures, might be impaired due to subtle deterioration of mental fitness. Neurophysiological measurement of mental fitness in such case could be beneficial for the person in understanding his or her mental state and put their decision-making in perspective, particularly compared to previous examples of lifetime decision-making as self or informant-assessed. It is conceivable that such a measure of suboptimal mental fitness might be useful in assessing the early stages of dementia of the various types including organic or neurological brain syndromes, Alzheimer's Dementia, or Traumatic Brain Injury, for example. It is also the case that such measures could be used to assess recovery, as a person's mental fitness improves, with the ultimate aim that dementia-related disorders could have reversible causes, if treated early and with appropriate and precise intervention.

To date, mental fitness has traditionally been assessed indirectly by psychological studies or physiological studies, and not directly by study of the brain. The lack of direct neurophysiological evaluation to assess mental fitness is largely due to the lack of reliable measures that can quantify key components of mental fitness, such as cognitive control. The present disclosure includes a system using quantitative EEG markers that can be used to assess the mental fitness state of a person before, during, and after a decision-making process, and thus in turn can help to optimize the person's decision-making.

### BRIEF SUMMARY

In broad concept, the present disclosure includes methods and systems, as well as components thereof, which assess a mental fitness state of a person using objective physiologic signals from the person. One or more sensors are arranged in proximity to the person's head to measure at least one electroencephalogram (EEG) signal of the person. The at least one EEG signal is analyzed by computing a power in the EEG signal in an EEG signal band, determining a characteristic of the computed power that, for the EEG signal band, is indicative of an emotional or cognitive state of the person, and assessing a mental fitness state of the person based on the determined characteristic. An assessment output is generated which reports the assessed mental fitness state of the person. In at least one implementation, the EEG signal contains oscillating theta waves, and a variance in timing of maximum power occurring in the theta waves around a determined average (mean) latency is calculated.

### BRIEF DESCRIPTION OF THE SEVERAL VIEWS OF THE DRAWINGS

FIG. 1 is a schematic drawing of a system, with subsystems and their components, according to at least one embodiment of the present disclosure.

FIG. 2A is a schematic drawing of a side view of a headset embedded with EEG sensors over labeled locations on a person's head.

FIG. 2B is a schematic drawing of a top view of the headset on a person's head indicating the locations of the EEG sensors as labeled in FIG. 2A.

FIG. 3A is a schematic drawing of a side view of a simplified headset embedded with an EEG sensor over a labeled location on a person's head.

FIG. 3B is a schematic drawing of a top view of the simplified headset on a person's head indicating the location of the EEG sensor as labeled in FIG. 3A.

FIG. 4A is a scatter plot of alpha desynchronization and its relationship to post-stimulus theta synchronization.

FIG. 4B describes an example of a congruent condition of an arrow flanker task which measures cognitive control and decision-making.

FIG. 4C describes an example of an incongruent (more difficult) condition of the arrow flanker task.

FIG. 5A is a graph illustrating an increase in probability of error when there is interference between ongoing theta signals, with a peak error rate at 140 ms (one theta cycle).

FIG. 5B is a scatter plot showing the predictive power of theta phase for reaction times (RTs). The correlation in this example is 0.88 and thus the theta phase almost perfectly predicts a behavioral measure (in this case RTs).

### DETAILED DESCRIPTION

Systems and methods disclosed herein determine the mental fitness state of a person using innovative physiologic signal inputs, such as EEG measures. Such systems and methods provide a user with an assessment of his or her mental fitness, gauges the user's progress in brain training, and can guide the user to make better decisions in an optimal state of mental fitness. Illustrative embodiments of the present disclosure are described by way of non-limiting example with reference to the figures.

Embodiments of the present disclosure use sensors, signal processing, and analytic algorithms to assess mental fitness before, during, and after a decision-making process, as well as measure mental fitness in the absence of decision making, and to quantify the relative influence of each stage of cognitive processing, for example, on the decision-making process. In the case of decision making, such assessment can help guide the decision maker to evaluate their decision-making ability in the moment and thereby choose to continue with the task at hand or to undertake or refrain from performing additional actions to optimize the decision-making process. The present disclosure can be applied to decision-making in general life issues and many other contexts, including marketing, consumer preference, investment and financial decisions, and other social and economic applications, for example. Assessment of decision making could also be used consistently to track an individual's decision making ability over time and in association with different states, e.g. early morning versus afternoon or evening, or with jet lag or lack of sleep.

Neuroscience has shown that most of cognition and memory utilization functions take place in the neocortex, the most highly evolved part of the brain. Emotion, on the other hand, is believed to be mostly processed in the midbrain region. There are certain neuro-pathways and neuro-processes, originating from the midbrain, that relate emotion and its effects to influence cognition and memory utilization processes in the neocortex. Neurophysiological processes related to cognitive control are known to emerge from the prefrontal and the parietal cortex. Such processes produce electrical potentials within the cortex, which can be measured on the scalp with temporal precision via electroencephalography (EEG) sensors, or electrodes, that are placed on the scalp. While embodiments disclosed herein focus on the impact of EEG signals related to cognitive control and how they contribute to mental fitness, it is the case that all electrophysiological activities in the brain may contribute to mental fitness, as well as other physiologic signals of the body. These signals can be assessed using the inventive principles disclosed herein.

While EEG measures may be used to evaluate emotion, attention, and cognitive functions, especially in the context of relating such to decision making, it has been challenging to quantify such parameters. In various embodiments, the present disclosure provides new and unique methods to use physiologic signals, such as EEG measures, to quantitatively assess the mental fitness state of a person—contributing from the person's cognitive execution, emotion, attention, and physical wellbeing—particularly as relating to decision-making. Such assessment can be used to gauge mental fitness as a wellness assessment, and also to assess the improvement of mental fitness as the person tries to improve such through physical, mental, and emotional wellness training—exercise, mental games, mediation, mindfulness training, as examples.

Physiologic signal sensors, such as EEG sensors, electrocardiogram (ECG) sensors, and sensors providing galvanic skin responses (GSR) have been used in the same traditional way for decades. Recently, there are several technology innovations that can be used to improve the use of these sensors. For example, such sensors and related electronics can be integrated, miniaturized, and embedded in polymer patches to make them flexible. Innovative electronics, with advanced signal capture and signal processing technologies, enable the sensors to be used without contacting the skin, requiring no conductive gel or pad. These advanced, flexible, non-contact sensors, such as those sensing EEG or ECG signals as examples, can be embedded in regular daily wear, to collect EEG or ECG data inconspicuously and comfortably from a person. With advances in mobile wireless technologies and cloud computing, signal processing and analysis can be done in increasingly complex and sophisticated ways which are more convenient to the user. Such mobile EEG applications include the use of artificial intelligence, brain-computer interface (BCI) and machine learning which have to date been applied successfully to non-mobile (laboratory) EEG and other neuroimaging data. These innovations open new possibilities in the use of physiological data, for example EEG and ECG data, in clinical and consumer applications. As an example, to date, most studies investigating emotion, cognition, and decision-making with EEG have been performed using the traditional laboratory-based EEG devices, which tend to be cumbersome, clinically-oriented and, by definition, non-mobile, which restricts data collection to a single setting.

A combination of mobile wearable EEG sensors with wireless technology that connects to a computer, mobile device, or computing cloud allows for powerful analytics that can elucidate an individual's level of cognitive control at any given time. Such information can then be used as an assessment, including self-assessment, to inform the quality of current or potential decision-making during normal-life jobs and functions. The present disclosure uses advanced technologies described to create a novel system to assess mental fitness, and to use such measures to optimize decision-making. As noted earlier, embodiments of the invention can be applied in a variety of contexts, including decision-making in life issues, marketing, consumer preference, investment and financial decisions, and other social and economic applications.

In various embodiments, disclosed herein is a method and system that assesses the mental fitness of a person and allows the use of these measures to assess mental fitness in general and specifically to also optimize decision-making processes, in the face of distractors, emotional states, or fatigue, for example. The assessment is performed by evaluating the person's neurophysiological measures quantitatively, with the option to be supplemented by other bio-physiologic measures. The outcomes of the assessment can be used to guide individuals in making more optimal and logical decisions.

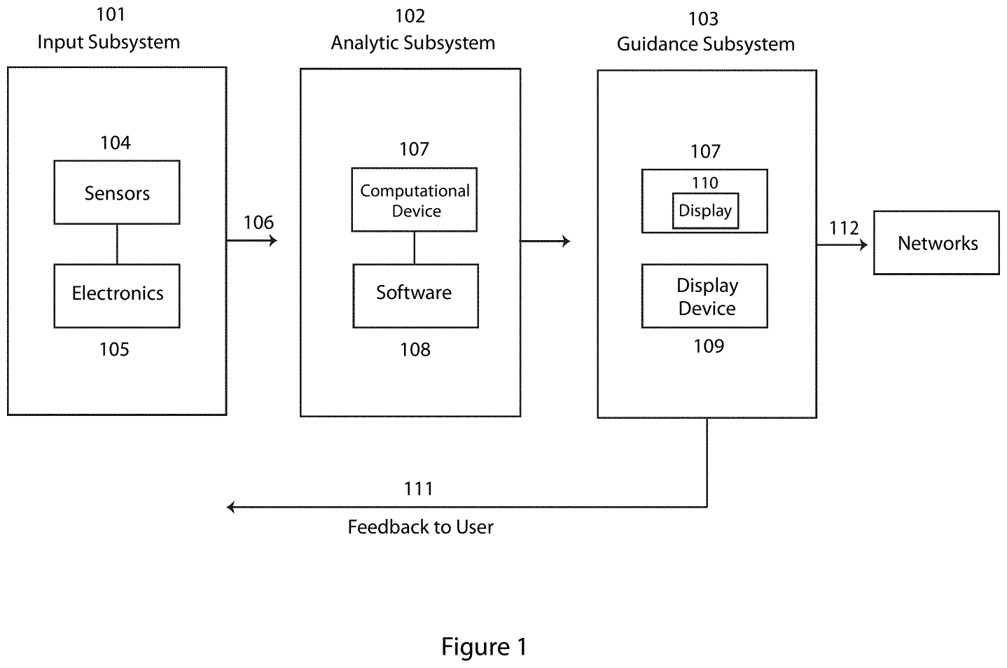

One example of a system for assessing mental fitness, and in turn assessment of decision-making, is illustrated in FIG. 1. The system comprises an input subsystem (101) that obtains a measurement of neurophysiological and bio-physiological information related to the mental fitness of a person as inputs; an analytic subsystem (102) that computes and analyzes the mental fitness state of the person, including during a decision-making process, based on the neurophysiological and bio-physiological information; and a guidance subsystem (103) that displays the analytic mental fitness state results and uses this to provide guidance on the decision-making process, with an option to provide a feedback mechanism to automate an optimization of the person's decision-making process. There is also an option to network with other users in making a group decision by assessing the individual mental fitness of persons in the group and collectively using the mental fitness state results of the individuals in the group to optimize the group's decision-making process.

The input subsystem (101) comprises sensors (104) that can measure neurophysiological and bio-physiological parameters of the person making a decision. These parameters can include but are not limited to, for example, EEG signals, ECG signals, heart rate, skin perspiration, galvanic skin response (GSR) signals, electromechanical signals, and/or optical pulse oximetry measures. The sensors can be contact or non-contact to the skin, requiring a conductive medium (e.g. electrolyte gel) or not, and be rigid or flexible. For example, the sensors may be embedded in patches or in clothing as wearable devices. The input subsystem also comprises electronic circuits (105) for signal processing and a power supply for example, with data transmission circuitry (106) providing wireless or hardwired communication options, e.g., to an analytic subsystem (102).

The analytic subsystem (102) comprises computational devices (107) such as, for example, a computer, a mobile device including a mobile tablet, a phone, a smart watch, or a computing cloud system, and software (108) operable by a computational device (107) for processing data including, for example, an analytic algorithm as described herein. The software (108) may comprise executable instructions that are stored in a memory communicatively coupled to a processor in the computational device (107). The processor executing the instructions implements the algorithm(s) to analyze the measured neurophysiological and bio-physiological parameters of the person received by the processor from the input subsystem (101) and produce one or more assessment data outputs that may be, for example, shown on a display also communicatively coupled to the processor. The analytic subsystem (102) may include software implementing an artificial intelligence algorithm that is teachable through machine learning to analyze the measured signal data received by the processor and suggest recommendations regarding the current decision made by the user based on information learned from the user's past decision-making patterns, the decisions made, and their outcome. The parameters to be computed and analyzed represent the mental fitness state of the person's neural processes during decision-making as indicated by physiological signals sensed by sensors positioned on or near the person.

The guidance subsystem (103) is communicatively coupled to the output of the analytic subsystem (102) and comprises a display device that is independent of (109), or as part of (110) the computational devices (107) being used as the analytic subsystem, to display relevant data collected and analytic results or recommendations to the user. The guidance subsystem (103) may also include wired or wireless connections (111) to a user interface that provides feedback to the person making the decision or to provide signals to one or more networks (112) of other users to optimize the decision-making process.

One example of the input subsystem (101) comprises electroencephalogram (EEG) sensors (104) being deployed in a standard configuration, using for example, 32, 64, 128 or 256 electrodes with placement utilizing standard 10-5, 10-10, or 10-20 systems.

Independent Component Analysis (ICA) acting as spatial filters can be used to process the EEG signals received from the input subsystem (101) to specify distinct information source(s) in the recorded multichannel data. This EEG setup can capture and provide extensive temporal and spatial information on the EEG data that is needed to index mental fitness, and related emotion and cognition measures. However, these types of standard high-number channels EEG devices have bulky, conspicuous headgear and typically require use with conductive gel or pads. In many instances, such an EEG setup is cumbersome, highly non-mobile, and labor intensive to use, and therefore is usually confined to clinical or laboratory settings.

It is valuable to further devise less complex and easier-to-use systems to assess mental fitness in general and to optimize decision-making processes applicable to the many economic, investment, financial commitments, consumer choices and daily-life decisions of a person. Optimizing a decision-making process comprises assisting a user (e.g., by instruction or other feedback) to make decisions preferably while in an optimal mental fitness state—with a determined level of cognitive control that can limit the emotion component contributing to the decision and increase the cognition and memory utilization effort component in order to make a more rational decision.

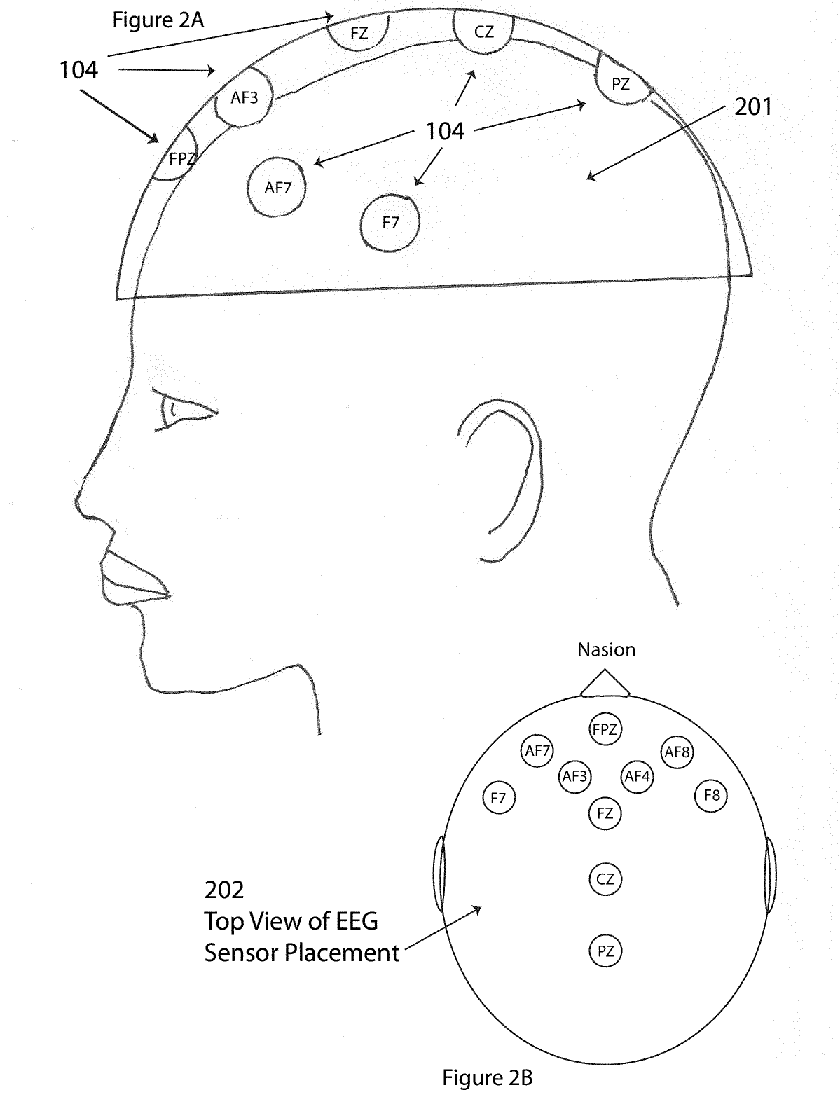

To enable use of a mental fitness assessment and decision-making system as described herein in a more realistic, mobile, daily-living setting, specific configurations of smaller numbers of EEG sensors may be placed over specific positions on the person's head. The sensors are designed and positioned to capture the desired temporal and spatial EEG information to derive mental fitness measures. In at least one non-limiting example, the input subsystem (101) comprises electroencephalogram (EEG) sensors (104), either contact or non-contact to the skin, requiring conductive medium or not, that are attached to the head of a person using a headset (201) as illustrated in FIGS. 2A and 2B, or using a wearable hat having a similar sensor configuration. The EEG sensors collect EEG signals from the brain over certain locations of the person's head in specific configurations, for example but not limited to the one illustrated in FIGS. 2A and 2B. For this example, ten EEG sensors (104), which can be contact or non-contact in type, are embedded in the headset (201) over specific positions of, for example, FPz, Fz, Cz, Pz, AF3, AF7, F7, AF4, AFB, and F8 as per standard defined locations (10-20 system) for each, covering the frontal and midline regions of the scalp, which are of specific interest in this example. Other example configurations may include EEG sensors, of different numbers, over different locations on the head, not limited to the frontal and midline regions as stated in the previous example, as designed to optimize the capture of relevant EEG information from the person. The EEG signals detected by the sensors (104) are transmitted to electronic circuits in the headset or hat to be processed, for example to be digitized and/or amplified, and transmitted through wire or wirelessly, to a computational device (107) of the analytic subsystem (102), for further processing and analysis with analytic software and algorithms. Machine learning or artificial intelligent algorithms, as noted earlier, may be implemented as part of the processes for analysis drawing from the user's past-history (sensed inputs and decision outcomes) in the course of helping the user optimize present decision-making.

Further specifications may be applied to the bands of EEG data to be studied in those specific locations, for example but not limited to, the theta and alpha bands. These specifications are used to optimize the EEG data collection in performing certain specific tasks, for example, measuring mental fitness, attention, and emotion, to be described in following sections. And since the number of EEG sensors needed are limited, as tailored to the specific need of EEG information location and frequency spectrum to be studied, the headgear for such can be much more non-obtrusive, inconspicuous, and tolerable. Using non-contact EEG sensors and wireless electronics with this example, the sensors may be embedded in regular headwear without the need for conductive gel or pads, making it acceptable as a regular wearable item to assess the user's mental fitness anytime, or while performing decision making in a daily living and work setting, outside of a laboratory or clinic.

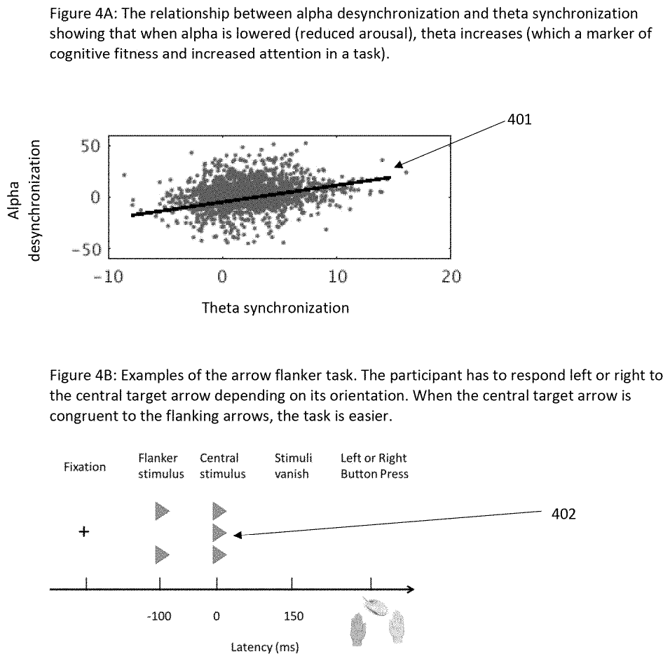

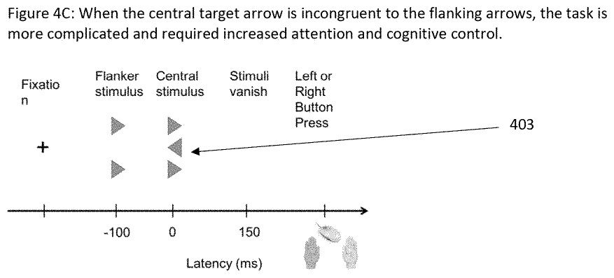

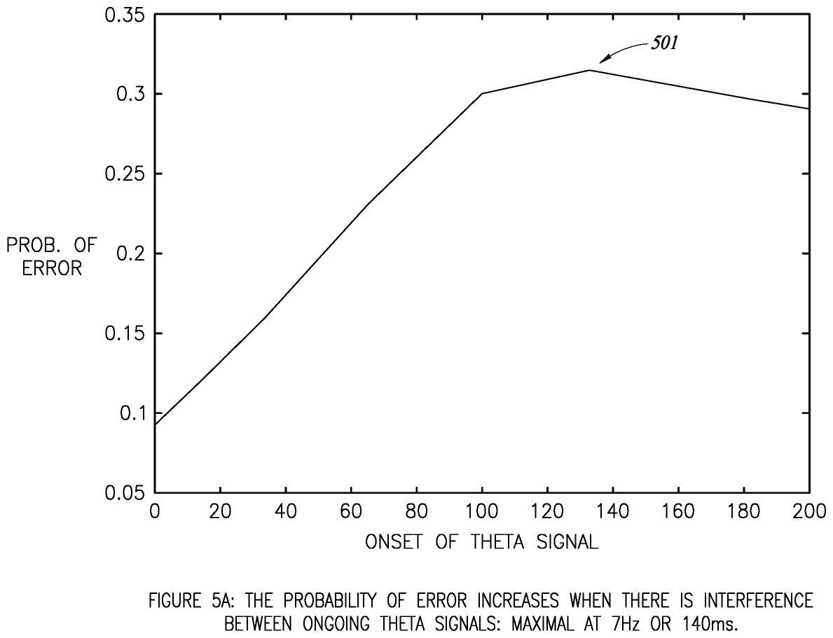

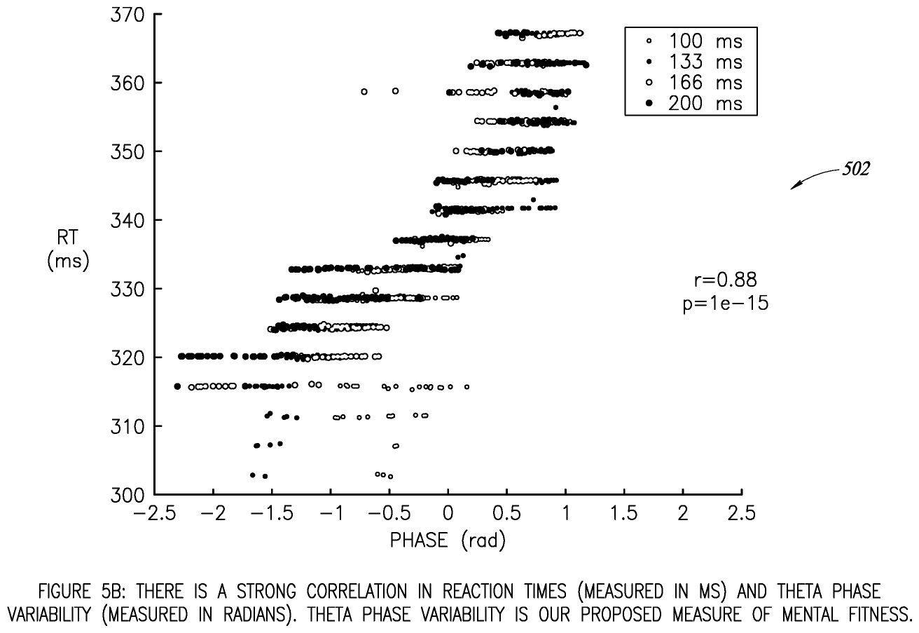

One iteration of the present disclosure measures and analyzes theta waves. EEG theta activity is defined as a signal where the maximal power (microvolts squared) in the EEG signal is between 4-8 Hz. In humans, the most typical theta wave signal is maximal at 7 Hz. Our work (in a series of studies that are presently unpublished) has established a relationship between a person's theta activity and the person's cognitive control and its impact on the person's attention/decision making. The premise of this work is rooted in discovering that a series of previously published studies indicated that power in theta wave signals clearly relates to conflict monitoring (an integral component of cognitive control and decision making), attention, and performance (errors and reaction times). In our work, we established that the theta signal impacted known EEG indices of conflict monitoring (an event-related potential (ERP) known as N2) and attention (an ERP known as P3). Further we established a relationship between the ability to switch off alpha band activity (8-13 Hz) and subsequent theta activity with alpha desynchronization (or switching off of alpha) on the y axis and theta synchronization (or switching on of theta) on the x axis, as illustrated in FIG. 4A. The line (401) shows that there is a significant and strong negative correlation between alpha and theta activity: the greater the power in the theta signal, the lower the power in the alpha signal. In relation to this patent disclosure, this correlation between alpha and theta activity is important as alpha activity is an index of arousal, e.g. being overcome with emotion can be viewed as over-aroused and therefore having a lack of alpha even during periods of rest (no action required) and conversely being fatigued can lead to an excess of alpha and therefore a state of under-arousal. The examination of alpha signals and theta signals can give a comprehensive picture of the mental fitness of an individual. Referring to FIGS. 4B and 4C, our work has shown that theta activity has a major impact on behavioral outcomes in a decision making task where the participant has to choose whether an arrow is pointing left or right in a flanker array that may be congruent (402) or incongruent to the central (target) arrow (403). More specifically, our work has shown that certain patterns of theta activity lead to poor decision making (i.e. increased errors) or more variable (i.e. unpredictable) reaction times during decision-making. Specifically, referring to FIG. 5A, the timing of the onset of the oscillation in the theta signal predicts number of errors (501) so that the probability of errors is maximal when the theta cycle is restarted instead of oscillating continuously (increased variability). Further, the timing of the theta cycle influences reaction times (502), as illustrated in FIG. 5B. Reaction times are a known indicator of mental fitness: the faster and more consistently one responds, the better the mental fitness. Slow and variable reaction times are associated with aging, tiredness, lack of cognitive control and arousal, and inattention. We propose to use direct markers via a mobile phone (or tablet) for example, to synchronize these markers with the EEG signal so that we measure theta signal peaks in relation to such markers in simple cognitive tasks being performed by the person. Our proposed index of mental fitness is the variance around an individual's average (mean) peak latency of the theta signal. As theta has a typical peak of 7 Hz, the timing of the peak should be between 120 milliseconds and 170 milliseconds. As will be described in greater detail below, there is a strong correlation in reaction times (measured in ms) and the timing of the onset of the theta signal (theta phase), in that when the theta phase, or onset, is more variable (measured in radians in FIG. 5B), reaction time is also more variable. Based on our knowledge of increased errors and more variable reaction times when theta activity is inconsistent in timing, specifically the onset of the theta signal, i.e. where the peak is not consistently every 140 ms, such variability can be considered a proxy of mental fitness. In other words, theta phase variability is used herein as a measure of mental fitness.

The analytic subsystem (102) comprises one or more computational devices, including for example a computing cloud, that provide powerful computational power for the analysis of EEG data, including the use of machine learning, transfer learning, and other forms of artificial intelligence capabilities. Computational analysis could involve training a machine over time to learn the peak theta power or coupling for an individual, which can inform ongoing assessments of the individual's decision making and/or mental fitness.

The analytic subsystem (102) can also provide information to the guidance subsystem (103) to advise the user on whether the person is approaching towards or deviating from an optimal mental fitness state. This could be presented as a bar depicting theta power during a guidance subsystem task. For example, this may involve a simple online fast Fourier transform that measures theta power as a moving average during the task. Such information may be especially helpful for the purpose of brain training, or during other efforts to improve one's mental function.

EEG data reflecting an individual's ‘typical’ theta, in one iteration as an example, can be defined by a short test of one to three minutes, which is sufficient for an accurate assessment of an individual's theta activity in that moment. Embodiments disclosed herein may offer visual and/or auditory options for such assessments. One visual example is to present an individual with a series of X and O stimuli, where the individual has to respond on their mobile phone, in the manner of a game, only to X stimuli and not to other letter (e.g., O) stimuli or to respond to the direction of arrows (as in FIG. 4B). An auditory example is where an individual is required to respond only to high frequency stimuli and not to low frequency stimuli. For most oscillations, 100 examples of the individual's peak theta activity are required to obtain a reliable average of oscillatory activity in the person's brain on a given day. The stimuli will be presented with required reaction times measured via the phone or tablet. The stimuli could be presented every 1-1.5 s. Therefore, an estimate of theta power could be achieved in three minutes or less. With this example, a theta peak is expected to be maximal at approximately 7 Hz, though this may have slight individual differences that can be estimated on an ongoing basis during the test/game. The lower the individual's peak theta activity in terms of power indicates variability in the person's theta waves, which in turn indicates poor attention, cognitive control, and mental fitness.

As noted, the short theta wave assessment can use audio or visual stimuli. In another example, an iteration of the test could present stimuli on a mobile phone or tablet or computer screen while the person is wearing the EEG headwear or headset. One iteration may use visual stimulus such as a simple reaction time task, during which the individual has to respond only to ‘X’ stimuli in a series of letters, ‘A’, ‘D’, ‘F’, ‘G’, etc., shown on a display. Another iteration may use auditory stimulus such that beeps are presented through headphones and the individual responds only to higher pitch beeps out of a series of lower pitched beeps. A number of tasks may be available to the individual via applications or apps on their computers, tablets, and/or phones, which may be the computational devices (107) used for the analytic subsystem (102) in the system shown in FIG. 1, or they can be separate devices linked to the computational devices (107).

Another iteration of the present disclosure involves several processes that may be used in an analysis of the emotional state of the person being studied, using EEG and possibly other physiological measures. One non-limiting example utilizes EEG measures of the alpha band power to estimate “frontal asymmetry,” which contrasts alpha signal power in the left frontal region with alpha signal power of the right frontal region. Traditionally, greater left-sided activation infers to more positive emotional valence (Tomarken 1990). Other studies have showed that frontal asymmetry indicating greater left-sided activation was linked to dispositional tendencies toward approach (vs. avoidance) (Sutton 1997) and anger emotion (Harmon-Jones 2006).

Without specifying any particular emotion, analysis of a person's EEG alpha band power, for evaluation of frontal asymmetry for example, identifying greater left-sided activation can indicate a greater emotion state as a “dimension”—the emotion dimension. In this example, a person's baseline level of frontal asymmetry in EEG alpha band power can be measured. The person then goes through a decision-making process and the level of frontal asymmetry in the person is evaluated during the process. The change in the level of the emotion dimension of the person can be derived and quantified by the measurement of frontal asymmetry.

There are other EEG measures to gauge the level of the emotion dimension that can also be utilized. For example, it has been shown that subjective scores of a person's emotional experiences significantly correlated with theta band power in the person's anterior and frontal midline region (Aftanas 2001). Firm support for the ability of EEG measures to quantify emotion comes from a study that shows a high classification rate (83.26%) for a range of emotions, including anger, disgust and fear (Murugappan et al. 2010) and another study that used a short EEG segment to correctly predict whether the participant was imagining a positive or negative emotional scenario, including love, awe, frustration and anger. This was based purely on spontaneous oscillatory EEG activity without stimulus event-locked responses and, through the use of a filtering algorithm, achieved an average accuracy of 71.3% (Kothe et al., 2013).

As specific EEG measures are developed and used to quantify a person's emotion dimension, the specific EEG sensor placements and EEG bands being studied can be specified and tailored to optimize the data collection in the most relevant, effective, and convenient way as described previously. As stated, one example may utilize non-contact EEG sensors embedded in a wearable hat or headset, without the need of conducting gel or saline pad, over specific regions of the person's head, for example the frontal and midline, to collect theta and alpha band EEG signals for processing and analysis to measure the emotional state of the person as illustrated in examples given. Alternatively, in another implementation, for example, a 64 channel standard EEG contact electrode system can be used to collect EEG signals from the person being studied, and the system may use ICA to process the desired specific information source locations and EEG bands to provide the needed information to measure the person's emotion state using, for example, alpha band frontal asymmetry and theta power from the frontal and midline regions, as described above. Based on the disclosed methods, further iterations of specific EEG sensor placements in the headwear and EEG assessments can be implemented, using specific information gained on source locations and EEG band markers relating to mental fitness, emotion, and cognition from multi-channel ICA studies and large scale mobile EEG studies. These examples leverage the information gained from such studies to tailor-make the headwear with even fewer EEG sensors, making the headwear even more user friendly for the consumer market.

A person's emotion state measured in the above-described examples is more of emotion as a dimension instead of a state of a specific emotion, such as fear, sadness, joy, or greed for example. Using emotion as a dimension simplifies the assessment and quantification of the person's emotion. Other examples can evaluate sensor signals, for example but not limited to EEG signals, to measure specific emotions such as pleasure or approach associated with left-sided activation in frontal asymmetry EEG studies; or similarly, specific emotions of negative valence or avoidance associated with right-sided activation. With analytic and artificial intelligent algorithms as described above, the person's specific emotional state, such as for example, fear, can be used to evaluate the person's decision-making in, for example equity investments, employing the analytic processes as illustrated by the examples described in the analytic sections below.

The cognition and/or memory effort dimension can likewise be evaluated by EEG studies. It has been demonstrated that EEG alpha and theta wave oscillations reflect cognitive and memory performance (Klimesch 1999). For example, good cognitive and memory performance is related to a tonic increase in alpha signal power but a decrease in theta signal power and, a large phasic (event-related) decrease in alpha signal power but increase in theta signal power. Another example is using ICA on high-density EEG data, e.g., as used in a study of simulated air traffic control tasks. In this case, five EEG independent component (IC) signals were found associated with specific neural substrates, specifically, the frontal, central medial, motor, parietal, and occipital areas (Dasari 2017). The theta band spectral power of the frontal IC and the alpha band spectral power of the four other ICs, were detected to be correlated to mental work load and effort level.

Another non-limiting example assesses cognition effort using a standard method of collecting EEG data with the standard placements of EEG electrodes, in conjunction with ICA for processing desired specific information source locations and EEG bands, to provide the EEG data to measure cognition and/or memory effort, e.g., utilizing the theta and alpha band spectral power measures of the ICs as described by Dasari above (Dasari 2017). In this example, the theta signal power peak in the frontal IC, and alpha signal power peak in the central medial, motor, parietal, and occipital ICs, are interpreted as being associated with increased cognitive effort.

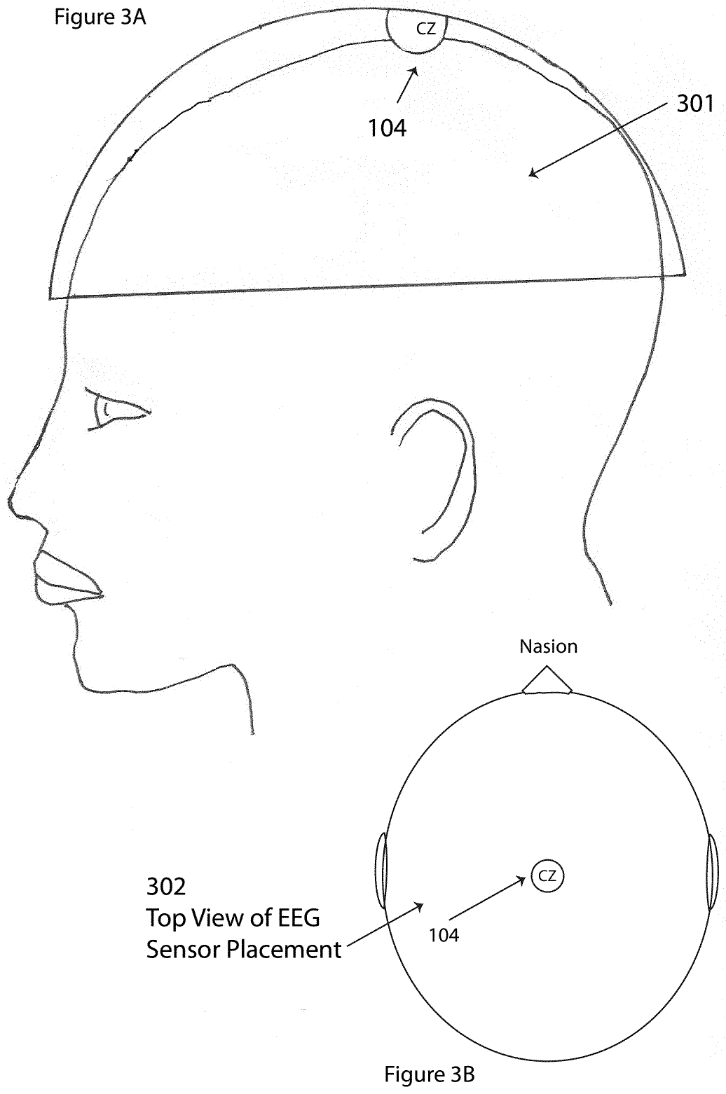

Another iteration of the present disclosure, as a non-limiting example, uses a simple headset with a few contact electrodes, or non-contact EEG sensors embedded in a wearable hat without the need of conducting gel or saline pad, over specific regions of the head, for example the frontal and midline, to collect theta band EEG signals for processing and analysis to measure cognitive and memory performance by studying the theta oscillations as described by McLoughlin et al. (McLoughlin 2014). Theta band activity projects to the frontal midline area of the scalp and thus placement of a contact or non-contact electrode at, for example, Cz (center of the head) or FCz (frontal midline) in any EEG headset, or specifically in a simplified EEG headset (301 in FIG. 3A, top view 302 in FIG. 3B) comprising at least one contact or non-contact electrode (104)—for example, in the Cz or FCz position—would be sufficient to measure phase of the theta band. Analysis of the signal would filter out activity other than that in the theta range—i.e. 4-8 Hz. A clear theta signal would thus be possible to identify, and the phase, or onset timing, of the theta signal could be assessed. If there is variability in phase onset greater than the average for that individual, the individual may be told that his/her mental fitness, and ability for decision making, is suboptimal. Similar to other wearable devices, the variability could be calibrated for each individual. As specific EEG measures are developed and used in particular embodiments to quantify cognitive and/or memory efforts, the specific EEG sensor placements and EEG bands being studied can be specified and tailored to optimize the data collection in the most relevant, effective, and convenient way as described above, with the objective to make the sensor embedded headgear or headwear more user friendly and tolerable by reducing the number and size of sensors and electronics in the design while serving specifically the intended functions of the embodiment being implemented.

The analytic subsystem (102) collects the EEG data from the input subsystem (101), then uses one or more analytic algorithms, with the option of including an artificial intelligent component that is teachable, e.g., by machine learning, to compute and analyze the EEG data to derive the mental fitness state, including measures reflecting cognitive effort and memory utilization, of the person's neural processes, e.g., during decision making.

There are individual differences in oscillatory peaks in EEG signals. Machine learning can provide a feedback loop to ensure that the assessment of mental fitness (e.g., theta phase consistency) is accurate for each individual user based on present and historical data for the user. For example, most adult humans have a theta peak power at 7 Hz; however, this may vary around 7 Hz and be anywhere between 5 and 8 Hz for different persons.

A machine learning system as described herein may improve accuracy of the assessment by using decision outcomes as further inputs to the system to tailor the analytic algorithm processing for particular individuals. A component of the system may include short online assessments with tasks using decision making stimuli. The outcomes of such assessments allow for calibration of the system for individual users.

Another iteration of the present disclosure uses EEG measures in brain-computer interface (BCI) systems. For example, a person's theta band activity could be trained over time and used to manipulate external systems, e.g., in terms of robotics—moving an arm left or right. Furthermore, using theta band measures in BCI allow embodiments of the present disclosure to determine when decisions, such as left or right, are made.

In some embodiments, the guidance subsystem (103) comprises a display device (109) that is independent of, or as part (110) of the computational devices (107) being used in the analytic subsystem, to display relevant data collected, analytic results, and/or recommendations for action by the user to improve the user's mental fitness state. The mental fitness state information may be, for example, displayed on the device. The user making a decision utilizes the provided information to evaluate any relevant mental fitness state component that has affected the decision made. Another option is that the user's mental fitness information is communicated via wired or wireless connections (112) to a network of other users utilizing the same type of devices and processes so as to optimize the decision-making process of the users functioning as a group, leveraging the effect of wisdom of crowds yet taking into account individual emotion considerations. For example, since each different user might have a different emotional reaction or be in a different mental fitness state when presented with a decision to be made, the aggregated mental fitness component measures in a group is usually diversified down as a contribution to the decision-making. However, in certain situations when “mob” or “herd” emotion mentality might set in, the system may differentiate such group emotion mentality by measuring an unusually high group emotion component based on an aggregate of measured emotion components of individual members of the group.

Another non-limiting example comprises a wired or wireless connection (111) to transmit guidance information to a user interface that provides feedback to the person making the decision or for the purpose of brain training. The guidance information may be in the form of electronic signals related to results derived from analysis performed by the analytic subsystem (102), e.g., mental fitness state results or processed signals from the source EEG information to be utilized to perform automated neuro-feedback to improve mental fitness or decrease the influence of emotion components in the user's decision-making process. An example utilizing this principle to help train a user's brain using a neuro-feedback process to optimize the user's mental fitness, including during a decision-making process, could be, as an example, as follows. The processed EEG signals relating to or representing the degree of mental fitness of the user may generate correlated graded audio tones that are output by the system via a speaker during a brain training session, or during the user's decision-making process. The user may then, for example, employ breathing exercises to decrease the emotion dimension as indicated by a change in the audio tone through neuro-feedback. In other words, illustrated as an example, the user uses a breathing exercise to optimize the user's mental fitness through neuro-feedback as reflected by hearing a decreasing audio tone indicating an improved mental fitness state. This process helps the user to optimize mental fitness and the user's current decision-making process, and further helps the user to apply such training to benefit future decision-making endeavors.

As for the types of decisions that can be assessed using the disclosed methods and systems, one non-limiting example involves binary decisions—yes or no; buy or sell; buy or hold; sell or hold; go or stay; and other similar decisions for example. The binary decision process would most likely allow generating relevant quantification of a person's mental fitness using study of the person's physiologic signals, such as EEG signals as described herein. Multiple choice/outcome decisions, such as choosing two stocks to invest in out of ten possible stocks, or choosing the best color for a dress out of five colors, in some situations can make the EEG information quantification less relevant. However, most multiple choice/outcome decisions can be reduced to relevant multiple binary decisions (with sub-decisions). Another non-limiting example uses multiple binary decisions, in which each decision allows more relevant quantification of the physiologic (such as EEG) information, to enable the construction of a decision tree or trees based on the binary sub-decisions needed to derive the final multiple choice/outcome decision.

While the foregoing detailed description involves obtaining and analyzing EEG signals, it is further understood that the systems and methods described herein may use other physiologic signals in addition to or in place of EEG signals. Such other physiologic signals may include electrocardiogram (ECG) signals, heart rate signal, measurements of perspiration, and/or measured galvanic skin conduction (GSR) parameters. For example, a computational device as described herein may analyze ECG signals obtained from ECG sensors placed on the person and identify increasing heart rate which usually associates with anxiety as indicative of the person's mental fitness state. Alternatively, or in addition, measurements of the person's heart rate, perspiration, and/or galvanic skin response may be obtained from respective sensors places on the person to identify increasing perspiration which can be associated with nervousness as indicative of the person's mental fitness state. The computational device may use such physiologic information to determine an assessment output that is relevant to the person's mental fitness state which may thereby enable optimization of the person's mental activity, e.g., in a decision-making process.

Various non-limiting aspects of the methods and systems described herein may be summarized, for example, as follows:

Aspect 1. A method and system to assess a person's mental fitness, and to optimize a person's decision-making process by assessing and utilizing one or more measures of the person's mental fitness state derived from objective physiologic inputs from the person, comprising:

sensors that measure one or more physiologic parameters of the person, for example but not limited to, electroencephalogram (EEG), electrocardiogram (ECG), heart rate, perspiration, or galvanic skin response (GSR) parameters, and

a computational device,

wherein the physiologic parameters measured by the sensors are transmitted to the computational device, wherein by using one or more analytic algorithms, the computational device quantifies physiologic information indicative of the mental fitness state of the person before and during a decision-making process, and such physiologic information is used by the computational device to determine one or more assessment outputs relevant to the person's mental fitness state, such as an assessment output that indicates a relative influence of the person's mental fitness state in the decision-making process, thereby enabling the person to optimize the decision-making process.

Aspect 2. The sensors in aspect 1 above can be contact or non-contact in nature.

Aspect 3. The sensors in aspect 1 above can be flexible or rigid.

Aspect 4. The sensors in any of the above aspects can capture a single type of physiologic information, for example EEG signal(s), or multiple types of physiologic information, for example EEG signal(s), ECG signal(s), and heart rate.

Aspect 5. The sensors in any of the above aspects can be embedded in a headgear to be worn over a person's head.

Aspect 6. The sensors in any of the above aspects can be non-contact sensors embedded in a regular headwear to be worn over the head without a conductive medium coupling the sensors to the head.

Aspect 7. The EEG sensors in any of the above aspects can be configured for placement on a person's head in standard EEG positions.

Aspect 8. The EEG sensors in any of the above aspects can be configured for placement in a specific pattern over the head designed to capture EEG signals from specific information locations and/or at specific EEG wave bands. This pattern can be determined in advance of constructing a wearable EEG headset, using high density EEG if desired.

Aspect 9. The physiologic information collected by the sensors in any of the above aspects can be wirelessly transmitted to the computational device.

Aspect 10. The computational device in any of the above aspects can include a computer, a mobile device including mobile tablet, a phone and/or smart watch, or computing cloud.

Aspect 11. The one or more analytic algorithms in any of the above aspects can include artificial intelligence such as machine-learning software.

Aspect 12. The decision to be made by the decision-making process in any of the above aspects can be binary in nature.

Aspect 13. The decision to be made by the decision-making process in any of the above aspects can be constructed using multiple binary decisions in the form of one or more decision trees.

Aspect 14. The assessment outputs from the computational device can be displayed on the computational device or on an independent display device.

Aspect 15. The assessment outputs from the computational device can be transmitted via a network and aggregated with assessment outputs from other users using the same type of computational device to perform group decision-making.

Aspect 16. The assessment outputs from the computational device can be used to provide neuro-feedback to the person being assessed and/or to other users of the system.

Aspect 17. The assessment outputs can be used by the computational device to gauge the person's mental fitness as a wellness assessment, also to assess an improvement of mental fitness as the person tries to improve such mental fitness through physical, mental, and emotional wellness training, including without limitation exercise, mental games, mediation, and mindfulness training, as examples.

Aspect 18. Determining the one or more assessment outputs may involve training a computing device to identify a peak theta band power or coupling for the person, which informs the assessment of the person's decision-making process and/or mental fitness.

The various embodiments and aspects described above can be combined to provide further embodiments. All of the U.S. patent applications and non-patent publications referred to in this specification and/or listed in the Application Data Sheet are incorporated herein by reference, in their entirety. Aspects of the embodiments can be modified, if necessary to employ concepts of the various applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Aftanas L I, Golocheikine SA Human anterior and frontal midline theta and lower alpha reflect emotionally positive state and internalized attention: High-resolution EEG investigation of meditation. Neuroscience Letters. 2001; 310:57-60.
Cavanagh J F, Zambrano-Vazquez L, Allen J J (2012): Theta lingua franca: a common mid-frontal substrate for action monitoring processes. Psychophysiology. 49:220-238.
Cavanagh J F, Figueroa C M, Cohen M X, Frank M J (2012): Frontal Theta Reflects Uncertainty and Unexpectedness during Exploration and Exploitation. Cereb Cortex. 22:2575-2586.
Dasari D, Shou G and Ding L. ICA-Derived EEG Correlates to Mental Fatigue, Effort, and Workload in a Realistically Simulated Air Traffic Control Task. Front. Neurosci. 2017; 11:297
Harmon-Jones E, Lueck L, Fearn M, Harmon-Jones C. The effect of personal relevance and approach-related action expectation on relative left frontal cortical activity. Psychological Science. 2006; 17(5):434-440.
Klimesch, W. EEG Alpha and Theta Oscillations Reflect Cognitive and Memory Performance: A Review and Analysis. Brain Research Reviews. 1999; 29: 169-195.
Kothe C, Makeig S, Onton J. Emotion Recognition from EEG during Self-Paced Emotional Imagery. International Conference on Affective Computing and Intelligent Interaction. Geneva, Switzerland. 2013. p. 855-858.
McLoughlin G, Palmer J A, Rijsdijk F, Makeig S: Genetic overlap between evoked frontocentral theta-band phase variability, reaction time variability, and attention-deficit/hyperactivity disorder symptoms in a twin study. Biol Psychiatry. 2014. 75:238-247.
Nigbur R, Ivanova G, Sturmer B (2011): Theta power as a marker for cognitive interference. Clin Neurophysiol. 122:2185-2194.
Priyanka P, Shah-Basak A, Deschamps T, Verhoeff P V, Jokel R, Meltzer J (2018): Spoentaneous oscillatory markers of cogniive status in two forms of demntia. Human Brain Mapp. 2018. In press.
Sutton S K, Davidson R J. Prefrontal brain asymmetry: A biological substrate of the behavioral approach and inhibition systems. Psychological Science 1997; 8(3):204-210.
Tomarken A J, Davidson R J, Henriques J B. Resting frontal brain asymmetry predicts affective responses to films. Journal of Personality and Social Psychology. 1990; 59:791-801.

1. A method for assessing a mental fitness state of a person using objective physiologic signals from the person, comprising:
arranging one or more sensors in proximity to the person's head to measure at least one electroencephalogram (EEG) signal of the person;
analyzing the at least one EEG signal by:
computing a power in the EEG signal in an EEG signal band;
determining a characteristic of the computed power that, for the EEG signal band, is indicative of an emotional or cognitive state of the person; and
assessing a mental fitness state of the person based on the determined characteristic; and
generating an assessment output that reports the assessed mental fitness state of the person.

2. The method of claim 1, wherein the at least one EEG signal contains oscillating theta waves in a frequency range between 4-8 Hz, and wherein determining the characteristic of the computed power includes:
identifying a timing of maximum power occurring in each theta wave of multiple theta waves;
determining an average latency of occurrence of the maximum power in the theta waves with respect to onset of the theta waves using the identified timing of the maximum power in each theta wave; and
calculating a variance in the timing of the maximum power in the theta waves around the determined average latency.

3. The method of claim 2, further comprising:
administering a cognitive task for the person to perform while the one or more sensors measure the at least one EEG signal;
measuring reaction times of the person while performing the cognitive task; and
determining a relationship of the measured reaction times with the onset of the theta waves.

4. The method of claim 2, further comprising implementing a machine learning process that uses historical measured EEG signals and decision outcomes of the person as inputs in a feedback loop to improve accuracy of calculating the variance in the timing of the maximum power in the theta waves.

5. The method of claim 1, further comprising administering a cognitive task for the person to perform while the one or more sensors measure the at least one EEG signal of the person, wherein the at least one EEG signal contains oscillating theta waves in a frequency range between 4-8 Hz, and wherein determining the characteristic of the computed power includes:
determining a maximum power occurring in each theta wave of multiple theta waves; and
using a variance in the amount of the maximum power in the multiple theta waves as the determined characteristic.

6. The method of claim 1, wherein the at least one EEG signal contains oscillating theta waves in a frequency range between 4-8 Hz and oscillating alpha waves in a frequency range of 8-13 Hz, and wherein analyzing the at least one EEG signal includes:
computing a power in the theta waves and a power in the alpha waves;
determining the characteristic of the computed power based on a comparison of the power in the theta waves with the power in the alpha waves; and
assessing the mental fitness state of the person based on an extent to which power in the theta waves is greater than power in the alpha waves.

7. The method of claim 1, wherein the at least one EEG signal contains alpha waves in a frequency range of 8-13 Hz, and
wherein analyzing the at least one EEG signal includes:
computing power in alpha waves that originate in a left frontal region of the person's head and a power in alpha waves that originate in a right frontal region of the person's head; and
determining the characteristic of the computed power based on a comparison of the power in the alpha waves originating in the left frontal region with the power in the alpha waves originating in the right frontal region.

8. The method of claim 1, wherein generating the assessment output further includes guiding the person in a decision-making process that involves multiple binary decisions constructed in a decision tree, and assessing the mental fitness state of the person at each decision of the multiple binary decisions.

9. The method of claim 1, further comprising optimizing a decision-making process by the person by providing the assessment output to the person as a neuro-feedback to guide the person to decrease the person's emotional influence on the decision-making process.

10. The method of claim 1, wherein the EEG signal band includes theta and alpha frequency bands, the method further comprising optimizing cognitive control in the person by using activity in the theta and alpha frequency bands, and a relationship between the activity in the theta and alpha frequency bands, to infer a cognitive state of the person including attention, concentration, and/or memory.

11. A system for assessing a mental fitness state of a person using objective physiologic signals from the person, comprising:
one or more sensors arrangeable in proximity to the person's head to measure at least one electroencephalogram (EEG) signal of the person; and
a computational device configured to analyze the at least one EEG signal by:
computing a power in the EEG signal in an EEG signal band;
determining a characteristic of the computed power that, for the EEG signal band, is indicative of an emotional or cognitive state of the person; and
assessing a mental fitness state of the person based on the determined characteristic,
wherein the computational device is further configured to generate an assessment output that reports the assessed mental fitness state of the person.

12. The system of claim 11, wherein the at least one EEG signal contains oscillating theta waves in a frequency range between 4-8 Hz, and wherein the computational device is configured to determine the characteristic of the computed power by:
identifying a timing of maximum power occurring in each theta wave of multiple theta waves;
determining an average latency of occurrence of the maximum power in the theta waves with respect to onset of the theta waves using the identified timing in each theta wave; and
calculating a variance in the timing of the maximum power in the theta waves around the determined average latency.

13. The system of claim 12, wherein the computational device is further configured to:
administer a cognitive task for the person to perform while the one or more sensors measure the at least one EEG signal;
measure reaction times of the person while performing the cognitive task; and
determine the onset of the theta waves based on the measured reaction times.

14. The system of claim 12, wherein the computational device implements a machine learning process using historical measured EEG signals and decision outcomes of the person in a feedback loop to improve accuracy of calculating the variance in the timing of the maximum power in the theta waves.

15. The system of claim 11, wherein the computational device is further configured to administer a cognitive task for the person to perform while the one or more sensors measure the at least one EEG signal, wherein the at least one EEG signal contains oscillating theta waves in a frequency range between 4-8 Hz, and wherein the computational device is configured to determine the characteristic of the computed power by:
determining a maximum power occurring in each theta wave of multiple theta waves; and
using a variance in the amount of the maximum power in the multiple theta waves as the determined characteristic.

16. The system of claim 11, wherein the at least one EEG signal contains oscillating theta waves in a frequency range between 4-8 Hz and oscillating alpha waves in a frequency range of 8-13 Hz, and wherein the computational device is configured to analyze the at least one EEG signal by:
computing a power in the theta waves and a power in the alpha waves;
determining the characteristic of the computed power based on a comparison of the power in the theta waves and the power in the alpha waves; and
assessing the mental fitness state of the person based on an extent to which power in the theta waves is greater than power in the alpha waves.

17. The system of claim 11, wherein the at least one EEG signal contains alpha waves in a frequency range of 8-13 Hz, and
wherein the computational device is configured to analyze the at least one EEG signal by:
computing a power in alpha waves that originate in a left frontal region of the person's head and a power in alpha waves that originate in a right frontal region of the person's head; and
determining the characteristic of the computed power based on a comparison of the power in the alpha waves originating in the left frontal region with the power in the alpha waves originating in the right frontal region.

18. The system of claim 11, wherein the one or more sensors include at least one contact or non-contact sensor that is embedded in a wearable hat or headset, and wherein in the case of a non-contact sensor, the non-contact sensor measures the at least one EEG signal without a conductive medium coupling the sensor to the person's head.

19. The system of claim 11, wherein the one or more sensors are arranged over the frontal and/or midline regions of the person's head.

20. The system of claim 11, wherein the computational device is further configured to guide the person in a decision-making process that involves multiple binary decisions constructed in a decision tree, and assess the mental fitness state of the person at each decision of the multiple binary decisions.

21. The system of claim 11, wherein the EEG signal band includes theta and alpha frequency bands, and wherein the computational device is configured to optimize cognitive control in the person by using activity in the theta and alpha frequency bands, and a relationship between the activity in the theta and alpha frequency bands, to infer a cognitive state of the person including attention, concentration, and/or memory.

22. The system of claim 11, wherein the computational device is further configured to optimize a decision-making process by the person by providing the assessment output to the person as a neuro-feedback to guide the person to decrease the person's emotional influence on the decision-making process.
